(19) United States
(12) Patent Application Publication
(10) Pub. No.: US 2012/0309838 A1
(43) Pub. Date: Dec. 6, 2012

(54) USE OF AGOMELATINE IN OBTAINING MEDICAMENTS INTENDED FOR THE TREATMENT OF OBSESSIVE-COMPULSIVE DISORDER (OCD)

(75) Inventors: Laurence Laigle, Maisons Laffitte (FR); Elisabeth Mocaer, Neuily Sur Seine (FR); Mark Millan, Le Pecq (FR)

(73) Assignee: Les Laboratoires Servier, Suresnes Cedex (FR)

(21) Appl. No.: 13/578,383

(22) PCT Filed: Feb. 10, 2011
(86) PCT No.: PCT/FR2011/000080

§ 371 Date: Aug. 10, 2012

(30) Foreign Application Priority Data
Feb. 11, 2010 (FR) 1000560

(51) Int. Cl. A61K 31/165; A61P 25/00

(52) U.S. Cl. 514/630

(57) ABSTRACT

The present invention relates to the use of agomelatine, or N-[2-(7-methoxy-1-naphthyl)ethyl]acetamide, in obtaining medicaments intended for the treatment of Obsessive-Compulsive Disorder (OCD).

The present invention relates to the use of agomelatine, or N-[2-(7-methoxy-1-naphthyl)ethyl]acetamide of formula (I):

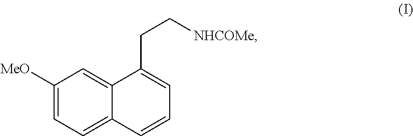

and also its hydrates, crystalline forms and addition salts with a pharmaceutically acceptable acid or base, in obtaining medicaments intended for the treatment of Obsessive-Compulsive Disorder (OCD).

Agomelatine, or N-[2-(7-methoxy-1-naphthyl)ethyl]acetamide, has the double characteristic of being, on the one hand, an agonist of receptors of the melatoninergic system and, on the other hand, an antagonist of the 5-HT_{2C} receptor. These properties provide it with activity in the central nervous system and, more especially, in the treatment of major depression, seasonal affective disorder, sleep disorders, cardiovascular pathologies, pathologies of the digestive system, insomnia and fatigue due to jet-lag, appetite disorders and obesity.

Agomelatine, its preparation and its use in therapeutics have been described in European Patent Specifications EP 0 447 285 and EP 1 564 202.

The Applicant has now found that agomelatine or N-[2-(7-methoxy-1-naphthyl)ethyl]-acetamide—and also its hydrates, crystalline forms and addition salts with a pharmaceutically acceptable acid or base—has valuable properties allowing it to be used in the treatment of Obsessive-Compulsive Disorder (OCD).

Obsessive-Compulsive Disorder (OCD) is a pathology defined by the presence of obsessions and compulsions. This pathology corresponds to perfectly defined criteria and constitutes a complete separate nosographic entity (300-3, Disorder—DSM IV—Diagnostic and Statistical Manual of Mental Disorders, 4^{th} Edition, American Psychiatric Association).

Obsessions are defined as recurrent thoughts, impulses or mental constructs which at some times are felt to be intrusive and inappropriate and which give rise to a large amount of distress. They are not merely excessive preoccupations with the problems of real life. The patient on the one hand makes an effort to ignore or repress them and on the other hand recognises that their origin lies within his or her own mental activity and that they are excessive or irrational.

Compulsions are repetitive behaviours or mental activities intended to neutralise or reduce the feeling of distress or to prevent a feared event or situation. Compulsions either bear no realistic relation to that which they are intended to neutralise or prevent or they are manifestly excessive.

The Obsessions or Compulsions give rise to marked feelings of distress and to the waste of a considerable amount of time. They especially interfere with socio-professional functioning and the day-to-day activities of the patient.

Obsessive-Compulsive Disorder is a chronic pathology which is not caused by the direct physiological effects of a substance. Its lifetime prevalence is of the order of 2 to 3% (Kaplan A. et al., Psychiatric Services, 2003, 54 (8)).

Currently there is no truly satisfactory treatment for Obsessive-Compulsive Disorder. Most frequently, patients are treated with the antidepressant clomipramine, a tricyclic antidepressant, or principally with serotonin reuptake inhibitors (SSRIs) in association with cognitive and behavioural therapy. However, SSRI treatments cause marked side-effects such as gastrointestinal disturbances e.g. nausea, anorexia, weight loss, sexual dysfunction or serotonin syndrome. Their efficacy is, moreover, not immediate but appears after a treatment period of 15 days to 3 weeks, and only 20% of patients respond to these treatments.

Accordingly, there still remains a real need for new treatments making it possible to improve the lives of patients suffering from Obsessive-Compulsive Disorder (OCD).

The Applicant has now found that, by virtue of its pharmacological properties and especially its excellent tolerability observed in clinical trials carried out on close to 3900 patients, agomelatine can be used in the treatment of Obsessive-Compulsive Disorder (OCD).

In particular, agomelatine does not have the side-effects associated with the customary psychotropic agents. Amongst those effects, the discontinuation syndrome observed on stopping treatment with the customary psychotropic agents is absent in the case of agomelatine, which makes it a treatment of choice in this indication.

The invention accordingly relates to the use of agomelatine, and also its hydrates, crystalline forms and addition salts with a pharmaceutically acceptable acid or base, in obtaining pharmaceutical compositions intended for the treatment of Obsessive-Compulsive Disorder (OCD).

The invention relates especially to the use of agomelatine obtained in the crystalline form II described in Patent Application EP 1 564 202 in obtaining pharmaceutical compositions intended for the treatment of Obsessive-Compulsive Disorder (OCD).

The pharmaceutical compositions will be presented in forms suitable for administration by the oral, parenteral, transcutaneous, nasal, rectal or perlingual route, and especially in the form of injectable preparations, tablets, sublingual tablets, glossettes, gelatin capsules, capsules, lozenges, suppositories, creams, ointments, dermal gels etc..

Besides agomelatine, the pharmaceutical compositions according to the invention comprise one or more excipients or carriers selected from diluents, lubricants, binders, disintegration agents, absorbents, colourants, sweeteners etc..

By way of non-limiting example there may be mentioned:
as diluents: lactose, dextrose, sucrose, mannitol, sorbitol, cellulose, glycerol,
as lubricants: silica, talc, stearic acid and its magnesium and calcium salts, polyethylene glycol,
as binders: magnesium aluminium silicate, starch, gelatin, tragacanth, methylcellulose, sodium carboxymethylcellulose and polyvinylpyrrolidone,
as disintegrants: agar, alginic acid and its sodium salt, effervescent mixtures.

The useful dosage varies according to the sex, age and weight of the patient, the administration route, the nature of the disorder and any associated treatments and ranges from 1 mg to 50 mg of agomelatine per 24 hours.

The daily dose of agomelatine will preferably be 25 mg per day, with the possibility of increasing to 50 mg per day.

Pharmaceutical Composition:

Formula for the preparation of 1000 tablets each containing 25 mg of active ingredient:

| | |
|---|---|
| N-[2-(7-methoxy-1-naphthyl)ethyl]acetamide | 25 g |
| Lactose monohydrate | 62 g |
| Magnesium stearate | 1.3 g |
| Povidone | 9 g |
| Anhydrous colloidal silica | 0.3 g |
| Cellulose sodium glycolate | 30 g |
| Stearic acid | 2.6 g |

Pre-Clinical Study

Pre-clinical studies were carried out using a model of Obsessive-Compulsive Disorder (OCD), confirming the potential of agomelatine for treatment of this pathology. Spontaneous marble burying in mice is a repetitive behaviour considered to be very relevant to OCD, and its inhibition suggests therapeutic activity in the treatment thereof (Witkin J. M., Current Protocols Neurosciences, 2008, Chapter 9, Unit 9.30). Following intraperitoneal administration at doses of 10, 40 and 80 mg/kg, agomelatine greatly reduced spontaneous marble burying in mice in dose-dependent manner, indicating therapeutic potential for the treatment of OCDs. The study was carried out as follows.

Male mice of the NMRI strain (Iffa-Credo, L'Arbresle, France), weighing 20-25 g on the day of the experiment, were placed individually in Macrolon boxes (30×18×19 cm) containing 5 cm of sawdust and covered with a perforated plexiglass plate. Twenty-four “cat's eye” glass marbles were evenly distributed on the sawdust at the periphery of the box. At the end of 30 minutes' free exploration, the animals were removed from the box and the number of buried marbles was counted. Agomelatine or the carrier (control) was injected 30 minutes before the start of the test.

The results obtained, given in terms of the number of marbles buried, are as follows:

Carrier: 20.2±0.6 (n=14)

Agomelatine 10 mg/kg: 19.2±1.3 (n=6)

Agomelatine 40 mg/kg: 15.3±3.0 (n=6)

Agomelatine 80 mg/kg: 4.6±1.9 (n=5)

Analysis of variance: F (3.33)=23.4 P<0.01. At the 40 and 80 mg/kg doses of agomelatine, P<0.05 vs the carrier (Dunnett's test).

The results obtained show statistically significant activity for agomelatine in a representative model of Obsessive-Compulsive Disorder.

Clinical Study

A clinical study comparing agomelatine with placebo was carried out in 80 out-patients more than 18 and less than 65 years in age, having a primary diagnosis of Obsessive-Compulsive Disorder according to the criteria of DSM-IV-TR. The patients must have, on entry into the study, a score of 20 or more on the Y-BOCS scale (Yale Brown Obsessive Compulsive Scale) and have been previously treated for their Obsessive-Compulsive Disorder with a serotonin reuptake inhibitor (SRI). The patients must have a depression severity score of less than 24 on the MADRS depression scale.

The study is a double-blind placebo-controlled study for a duration of treatment of 16 weeks. The patients are randomised either into the placebo group or into the 25 mg agomelatine group with the possibility of increasing the dose of agomelatine to 50 mg in the event of failure to respond after 8 weeks of treatment (criterion for failure to respond: less than 20% reduction in the total Y-BOCS score).

The main criterion for the assessment of efficacy is the reduction in the total score on the Y-BOCS scale. The other assessment criteria for evaluating the severity of the obsessive and/or compulsive state are the scores on the NIMH-OC (National Institute of Mental Health Obsessive-Compulsive scale) and CGI-S (Clinical Global Impression-Severity), as well as the improvement in that state by the CGI-I (Clinical Global Impression-Improvement). The presence of depressive symptoms and their course over time are analysed by the MADRS depression scale at the start of treatment and after 16 weeks of treatment.

Response is defined as a 35% reduction in the total score on the Y-BOCS scale and a score of 1 or 2 on the CGI-I. Remission is defined by a score of less than or equal to 10 on the Y-BOCS scale and of less than or equal to 2 on the CGI-S.

The results observed confirm the efficacy of agomelatine in the treatment of Obsessive-Compulsive Disorder (OCD) and also its good acceptability profile.

1-6. (canceled)

7- A method of treating Obsessive-Compulsive Disorder (OCD), in a subject in need thereof, comprising administration of an effective amount of agomelatine or N-[2-(7-methoxy-1-napthyl)ethyl]acetamide, or a hydrate, crystalline form or addition salt thereof with a pharmaceutically acceptable acid or base.

8- The method of claim 7, wherein the agomelatine is in crystalline form II.

9- The method of claim 7, wherein the agomelatine, or a hydrate, crystalline form or addition salt with a pharmaceutically acceptable acid or base, is administered in combination with one or more pharmaceutically acceptable excipients.

10- The method of claim 8, wherein the crystalline form II of agomelatine is administered in combination with one or more pharmaceutically acceptable excipients.
